(19) United States
(12) Patent Application Publication
(10) Pub. No.: US 2022/0160418 A1
(43) Pub. Date: May 26, 2022

(54) CONDUCTIVE ELECTRODE FOR ELECTROSURGICAL HANDPIECE

(71) Applicant: In Sang CHOI, Anyang-si (KR)

(72) Inventor: Bo-Hwan CHOI, Asan-si (KR)

(73) Assignee: In Sang CHOI, Anyang-si (KR)

(21) Appl. No.: 17/437,068

(22) PCT Filed: Dec. 23, 2019
(86) PCT No.: PCT/KR2019/018283

§ 371 Date: Sep. 8, 2021

(30) Foreign Application Priority Data
Apr. 9, 2019 (KR) 10-2019-0041566

(51) Int. Cl. A61B 18/14

(52) U.S. Cl. CPC A61B 18/148; A61B 2018/00077

(57) ABSTRACT

The present invention relates to a conductive electrode which is used for an electrosurgical handpiece and, more particularly, to a conductive electrode for an electrosurgical handpiece, wherein the conductive electrode is split into two pieces, and thus not only a general surgery can be performed as in the conventional electrosurgical handpiece, but also a precise surgery can be performed by even an unskilled surgeon, and the fatigue of an operating surgeon can be reduced.

### TECHNICAL FIELD

The present invention relates to a conductive electrode for an electrosurgical handpiece, and more particularly, to a conductive electrode for an electrosurgical handpiece, in which the conductive electrode is split into two pieces, thereby allowing a user to select and perform any one among a general surgery as in the conventional electrosurgical handpiece and a precise surgery.

### BACKGROUND ART

An iron surgical scalpel must cause damage only to tissues when cutting the tissues in order to cut cleanly. However, the iron surgical scalpel has not coagulation effect. That is, when tissues are cut, bleeding continues till cutting ends or the incision part is coagulated natural.

Electrosurgery is a surgical method of cutting, ablating or cauterizing tissues of a patient using high frequency (radio frequency) electric energy.

Vibration occurs in cells by electric energy supplied through an electrode, thus temperature in the cells rises to heat tissues.

When temperature in the cells reaches about 60° C., cell death starts, and when temperature rises to 60° C. to 99° C., tissue drying (dehydration) and protein coagulation progress. When temperature in the cells reaches 100° C., volume expansion of the cells and vaporization occur. In this process, tissues are cut or cauterized.

Such electrosurgery uses high frequency electric current in order to cut and coagulate tissues, and cutting using an electrosurgical device generates heat during tissue cutting by high frequency electric current so as to provide remarkable coagulation effect.

However, electrosurgical cutting generates arc together with high fever while an air insulation layer is destroyed by a conductive electrode and incomplete contact of tissues. Because the tissues burn by the arc, the patient may get burned, the tissues may be carbonized, and a blade may be contaminated.

Moreover, while the tissues are carbonized by the arc, smog may be generated as illustrated in FIG. 1. It has been known that smog has a bad influence on an operating surgeon's and a patient's health.

### DISCLOSURE

### Technical Problem

Accordingly, the present invention has been made in an effort to solve the above-mentioned problems occurring in the prior arts, and it is an object of the present invention to provide a conductive electrode for an electrosurgical handpiece, in which can allow a user to select and perform any one among a general electrosurgical surgery and a precise electrosurgery, reduce electrode contamination, reduce burn of a patient, and minimize generation of smog.

### Technical Solution

To achieve the above objects, the present invention provides a conductive electrode for an electrosurgical handpiece, which is coupled to a handpiece used for electrosurgery, the conductive electrode including: a first blade formed of a conductive material in a plate shape; a second blade formed of a conductive material in a plate shape; and a gap formed between the first and second blades so that the first and second blade are spaced apart from each other at a predetermined gap.

In this instance, the conductive electrode further includes: a first plug formed to be extended from the rear end of the first blade and inserted and coupled into a handpiece; and a second plug formed to be extended from the rear end of the second blade and inserted and coupled into a handpiece.

Moreover, a front end portion of the gap has a split angle which is inclined at a predetermined angle to an axial line of the conductive electrode.

Furthermore, the split angle is formed to be inclined in the direction of the first blade.

Additionally, the split angle is within a range exceeding 0° but not exceeding 120° in the direction of the first blade.

In addition, the first blade and the second blade are coated with an insulating material.

### Advantageous Effects

The conductive electrode according to an embodiment of the present invention which is split into a first blade and a second blade can allow a user to perform a general electrosurgery by conducting high frequency electric energy to both of the first and second blades or to perform a precise surgery by conducting high frequency electric energy only to the first blade.

Furthermore, the conductive electrode for an electrosurgical handpiece according to an embodiment of the present invention can reduce the degree of fatigue of an operating surgeon by reducing the doctor's tension since conducting high frequency electric energy only to the first blade in order to perform a precise electrosurgery.

Additionally, the conductive electrode for an electrosurgical handpiece according to an embodiment of the present invention can reduce contamination of the blades and a patient's burn at the time of the precise surgery, and does not generate smog which has a bad influence on the operating surgeon's and the patient's health.

### DESCRIPTION OF DRAWINGS

FIG. 1 is a view illustrating the configuration of an electrosurgical instrument.

FIG. 2 is a perspective view illustrating a handpiece among electrosurgical instruments.

FIG. 3 is an exploded perspective view illustrating a state where a conductive electrode according to an embodiment of the present invention is separated from the handpiece.

FIG. 4 is a perspective view illustrating the conductive electrode of the present invention.

FIG. 5 is a view illustrating a state where a coated layer and an insulator are removed from the conductive electrode of the present invention.

FIG. 6 is a view illustrating connection of the conductive electrode and a control unit.

FIG. 7 is a view illustrating a state where a precise electrosurgery is performed using a first blade of the conductive electrode of the present invention.

FIG. 8 is a view illustrating a state where a general electrosurgery is performed using the first blade and a second blade of the conductive electrode of the present invention.

FIG. 9 is a view illustrating a state where a split angle is formed at the front end of the conductive electrode of the present invention.

### MODE FOR INVENTION

Hereinafter, preferred embodiments of the present invention will now be described in detail with reference to the attached drawings, in which like reference numbers denote corresponding parts throughout the drawings.

The terms “comprising” and “including” in the discussion directed to the present invention and the claims are used in an open-ended fashion and thus should be interrupted to mean “including”, but not limited thereto.

A conductive electrode 100 which is used in a handpiece of an electrosurgical instrument is configured to cut, ablate or cauterize tissues using high frequency electric energy supplied to the conductive electrode 100.

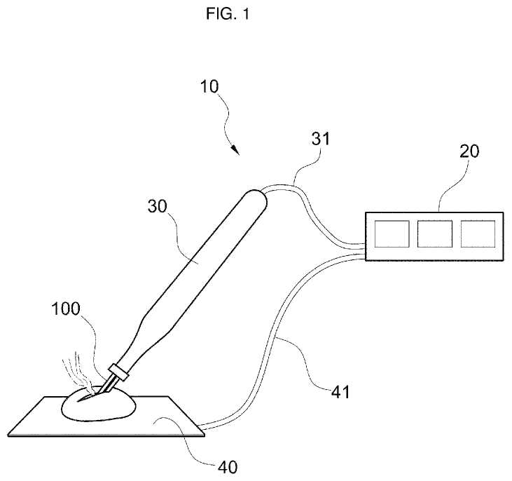

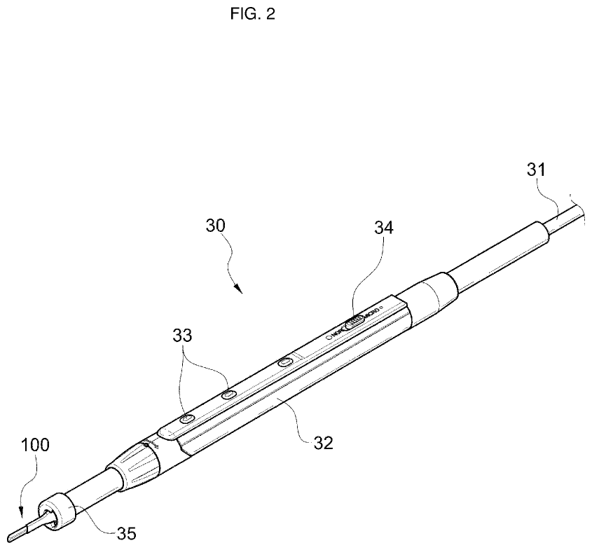

The handpiece 30 according to an embodiment of the present invention is a monopolar electrosurgical instrument, and as illustrated in FIGS. 1 and 2, the conductive electrode 100 is coupled to the front of the handpiece 30, which is a part that an operating surgeon grasps with the hand, a grounding pad 40 is grounded to a patient, and the handpiece 30 and the grounding pad 40 are respectively connected to a control unit 20, which generates high frequency, through cables 31 and 41.

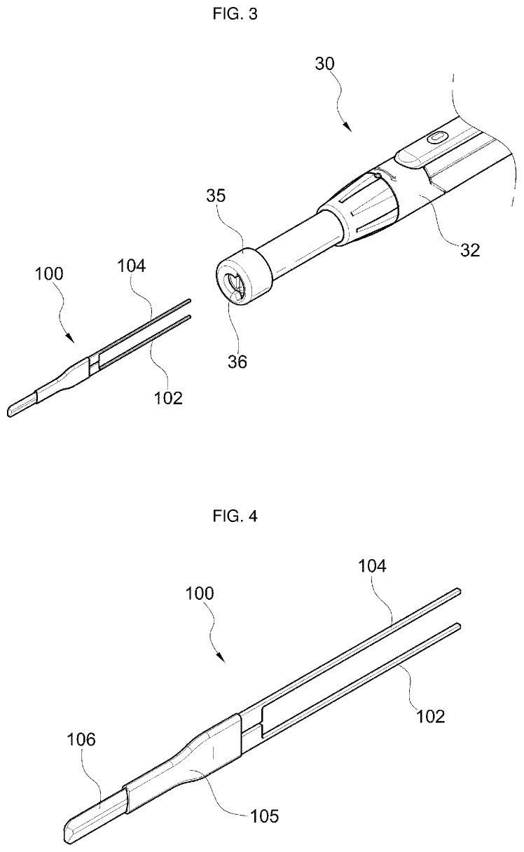

As illustrated in FIG. 3, the conductive electrode 100 has a first plug 102 and a second plug 104 formed at the rear of the conductive electrode 100. The first plug 102 and the second plug 104 are inserted into an insertion hole 36 of a holder 35 formed at the front of the handpiece 30, and the high frequency electric energy generated in the control unit 20 is supplied through the cable 31.

An operating surgeon can perform a surgery by the high frequency electric energy transferred to the conductive electrode 100, and in this instance, the conductive electrode 100 is formed in a long plate shape so that the operating surgeon can easily perform cutting, ablation or cauterization of tissues, and especially, cutting of tissues is carried out by an edge portion of the electrode of the long plate shape.

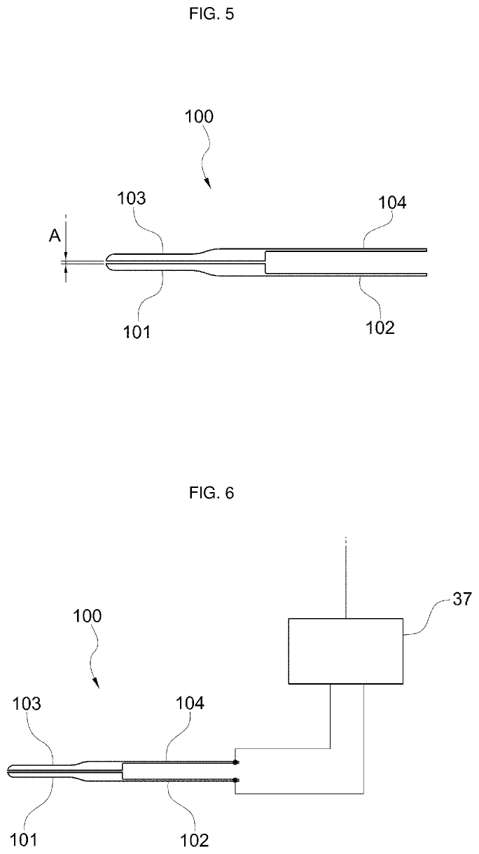

As illustrated in FIG. 5, the conductive electrode 100 according to the embodiment of the present invention includes the first plug 102 extending from the rear of a first blade 101 formed of conductive metal in a plate shape and the second plug 104 extending from the rear of a second blade 103 formed of conductive metal in a plate shape.

The first blade 101 and the second blade 103 are spaced apart from each other at a predetermined gap (A).

Moreover, as illustrated in FIG. 4, the first blade 101 and the second blade 103 which are spaced apart from each other at the predetermined gap (A) respectively have coated layers 106 formed at front portions of the first blade 101 and the second blade 103, and the coated layer 106 is formed by coating agent of a ceramic material applied thereto, so that the first blade 101 and the second blade 103 are fixed while keeping the predetermined gap (A).

Furthermore, insulation between the first blade 101 and the second blade 103 is maintained by the coated layers 106.

Preferably, rear portions of the first and second blades 101 and 103 having the coated layers 106 are wrapped with insulators 105, so that the first and second blades 101 and 103 are not exposed as illustrated in FIG. 2 when the conductive electrode 100 is inserted into the insertion hole 36 of the holder 35 of the handpiece 30 as illustrated in FIG. 3.

As described above, the conductive electrode 100 split into the first and second blades 101 and 103 is inserted into the insertion hole 36 of the holder 35 of the handpiece 30 to be fixed to the handpiece. As illustrated in FIG. 6, when the conductive electrode 100 is inserted into the insertion hole 36 of the holder 35 of the handpiece 30, as illustrated in FIG. 6, the first plug 102 and the second plug 104 are electrically connected to the control unit 37 of the handpiece 30.

As illustrated in FIG. 2, the control unit 37 has an operation button 33 and a selection lever 34 formed on a case 32 of the handpiece 30. The operation button 33 serves to supply the high frequency electric energy generated in the control unit to the conductive electrode 100 or to cut off supply of the high frequency electric energy to the conductive electrode 100, and the selection lever 34 serves to selectively supply the high frequency electric energy supplied to the conductive electrode 100 to the first blade 101 and the second blade 103.

The selection lever 34 allows the operating surgeon to select a ‘NOR’ mode, a ‘MICRO’ mode, and a ‘MEDIUM’ mode. The high frequency electric energy is supplied to all of the first blade 101 and the second blade 103 in the ‘NOR’ mode, is supplied only to the first blade 101 in the ‘MICRO’ mode, and is supplied only to the second blade 103 in the ‘MEDIUM’ mode.

The first blade 101 of the conductive electrode 100 according to the embodiment of the present invention is a part which first gets in contact with tissues when the operating surgeon performs a surgery while grasping the handpiece 30 with the hand, and the second blade 103 is a part which is inserted into the tissues depending on the first blade 101.

When the operating surgeon puts the selection lever 34 of the handpiece 30 in the ‘MICRO’ mode and presses the operation button 33 of the handpiece 30 in order to supply high frequency electric energy to the conductive electrode 100, the high frequency electric energy is supplied only to the first blade 101 but is not supplied to the second blade 103.

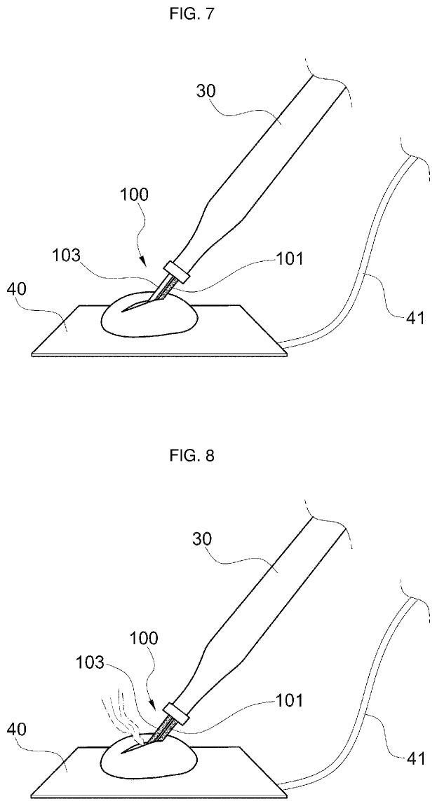

In the above state, when the operating surgeon puts the conductive electrode 100 to the tissues, as illustrated in FIG. 7, the first blade 101 first gets in contact with the tissues, and the tissues are cut by the high frequency electric energy.

The second blade 103 following the first blade 101, which advances while cutting the tissues, does not generate arc even though getting in incomplete contact with the tissues since the high frequency electric energy is not supplied in the ‘MICRO’ mode. Because arc is not generated, there is no carbonization or burning of the tissues and there is no generation of smog.

As described above, when the operating surgeon performs an electrosurgery in the ‘MICRO’ mode using the conductive electrode 100, since the first blade 101 gets in contact with the tissues and the second blade 103 is not supplied with high frequency electric energy, as illustrated in FIG. 7, there is no generation of arc, carbonization or burning of tissues, and generation of smog during the surgery.

When the operating surgeon performs a surgery in the ‘MICRO’ mode using the handpiece 30, speed that the conductive electrode 100 cuts, ablates or cauterizes tissues is reduced. However, the conductive electrode according to the embodiment of the present invention can perform a precise surgery using less high frequency electric energy without carbonization or burning of the tissues and without generation of smog.

Therefore, when the operating surgeon performs a surgery only using the first blade 101 of the conductive electrode 100 according to the embodiment of the present invention, an unskilled surgeon can perform a precise electrosurgery since excessive cutting, ablation or cauterization is prevented.

Moreover, because even the unskilled surgeon can perform a precise surgery in the ‘MICRO’ mode to supply high frequency electric energy only to the first blade 101, the conductive electrode according to the embodiment of the present invention can reduce the degree of fatigue.

In case that an operating surgeon performs a general electrosurgery, such as fast cutting, ablation or cauterization of lots of tissues, when the operating surgeon puts the selection lever 34 of the handpiece 30 in the ‘NOR’ mode and presses the operation button 33 of the handpiece 30, high frequency electric energy is supplied to all of the first blade 101 and the second blade 103 of the conductive electrode 100.

In the above state, when the operating surgeon puts the conductive electrode 100 to the tissues, as illustrated in FIG. 8, while the first blade 101 gets in contact with the tissues, cutting of the tissues is started by the high frequency electric energy. After that, the conductive electrode 100 is inserted into the tissues, and the second blade 103 following the first blade 101 is inserted into the tissues. Thus, the tissues are cut rapidly in a wide scope.

In this instance, the first blade 101 cuts the tissues in perfect contact with the tissues, but the second blade 103 cuts the tissues in imperfect contact with the tissues. So, arc is generated and smog is also generated as illustrated in FIG. 8, but the operating surgeon can rapidly perform an electrosurgery using the first and second blades 101 and 103.

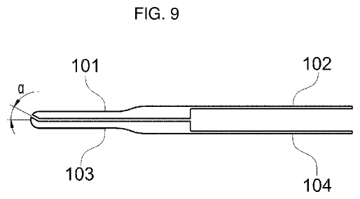

As described above, the conductive electrode 100 according to the embodiment of the present invention is split into the first blade 101 and the second blade 103 spaced apart from each other at the predetermined gap (A). In this instance, as illustrated in FIG. 9, it is preferable that the gap (A) formed between the front ends of the first and second blades 101 and 103 be inclined at a predetermined angle to a longitudinal axial line of the first and second blades 101 and 103.

Hereinafter, the inclined angle of the front gap (A) between the first and second blades 101 and 103 is called a ‘split angle (α)’.

Because the part of the conductive electrode 100 first getting in contact with the tissues at the time of an electrosurgery is the first blade 101, as described above, the split angle (α) formed at the front ends of the first and second blades 101 and 103 is an inclination angle within a range exceeding 0° but not exceeding 120° in the direction of the first blade 101.

As described above, because the inclined split angle (α) is formed at the gap (A) between the front ends of the first and second blades 101 and 103, the drawn line length of the first blade 101 is adjusted, and so, the operating surgeon can perform a precise surgery better in the ‘MICRO’ mode.

When the split angle (α) is 120°, the drawn line length of the first blade 101 becomes shorter than a case that the split angle (α) is 0°. So, the operating surgeon can perform the precise surgery more accurately when the split angle (α) is 120°.

Because the adjustment of the drawn line length of the first blade 101 when the split angle (α) is 0° has no meaning, it is preferable to make the split angle (α) exceed 0°.

Furthermore, when the operating surgeon performs the surgery while grasping the handpiece 30 with the hand, an angle formed between the conductive electrode 100 and the tissues is generally 120°. So, it is preferable that the split angle (α) do not exceed 120°.

As described above, when the gap (A) between the front ends of the first and second blades 101 and 103 is formed at the inclined split angle (α) relative to the longitudinal axial line of the first and second blades 101 and 103, the surgeon can perform a surgery in the ‘MEDIUM’ mode besides the ‘MICRO’ mode and the ‘NOR’ mode.

In the ‘MEDIUM’ mode, high frequency electric energy is supplied only to the second blade 103 but is not supplied to the first blade 101.

When the operating surgeon puts the selection lever 34 of the handpiece 30 in the ‘MEDIUM’ mode and presses the operation button 33 of the handpiece 30, high frequency electric energy is supplied only to the second blade 103 but is not supplied to the first blade 101.

In the ‘MEDIUM’ mode, the second blade 103 first gets in contact with the tissues, and next to the second blade 103, the first blade 101 is inserted into the tissues.

As described above, because the split angle (α) is formed to be inclined in the direction of the first blade 101, the drawn line of the second blade 103 gets longer than the drawn line of the first blade 101.

Therefore, the speed to cut, ablate or cauterize tissues in the ‘MEDIUM’ mode that the second blade 103, which has the drawn line longer than that of the first blade 101, gets in contact with the tissues earlier than the first blade 101 is faster than that in the ‘MICRO’ mode that the first blade 101, which has the drawn line shorter than that of the second blade 103, that is, the speed to cut, ablate or cauterize tissues in the ‘MEDIUM’ mode is almost the same as the ‘NOR’ mode that the speed to cut, ablate or cauterize tissues is fast.

Additionally, because high frequency electric energy is not supplied to the first blade 101 following the second blade 103 when the operating surgeon performs an electrosurgery in the ‘MEDIUM’ mode, arc is not generated even though the first blade 101 get in imperfect contact with the tissues. Because air is not generated, there is no carbonization or burning of the tissues and there is no generation of smog.

That is, when the operating surgeon performs a surgery in the ‘MEDIUM’ mode of the handpiece 30, the conductive electrode 100 can cut, ablate or cauterize tissues as nearly fast as that in the ‘NOR’ mode, so that the operating surgeon can perform a precise surgery without carbonization or burning of the tissues and generation of smog.

As described above, the conductive electrode 100 according to the embodiment of the present invention which is split into the first blade 101 and the second blade 103 can allow the surgeon to perform a general electrosurgery by conducting high frequency electric energy to both of the first and second blades 101 and 103 or to perform a precise surgery by conducting high frequency electric energy only to the first blade.

Furthermore, the conductive electrode for an electrosurgical handpiece according to the embodiment of the present invention can reduce the degree of fatigue of the operating surgeon by reducing the doctor's tension since conducting high frequency electric energy only to the first blade 101 or the second blade 103 in order to perform a precise electrosurgery.

Additionally, the conductive electrode for an electrosurgical handpiece according to an embodiment of the present invention can reduce contamination of the blades and a patient's burn at the time of the precise surgery, and does not generate smog which has a bad influence on the operating surgeon's and the patient's health.

The conductive electrode according to an embodiment of the present invention is configured to be suitable for a monopolar electric circuit, but may be configured to be suitable for a bipolar electric circuit.

The technical thoughts of the present invention have been described hereinafter.

It is to be appreciated that those skilled in the art can change or modify the embodiments from the above description in various ways. Although it is not clearly illustrated or described herein, it is to be appreciated that those skilled in the art can change or modify the embodiments from the above description in various ways without departing from the scope and spirit of the present invention and such changes and modifications belong to the scope of the present invention.

While the present invention has been described with reference to the particular illustrative embodiments, it is not to be restricted by the embodiments but only by the appended claims.

### INDUSTRIAL APPLICABILITY

As described above, the conductive electrode 100 according to the embodiment of the present invention which is split into the first blade 101 and the second blade 103 can allow the surgeon to perform a general electrosurgery by conducting high frequency electric energy to both of the first and second blades 101 and 103 or to perform a precise surgery by conducting high frequency electric energy only to the first blade.

Furthermore, the conductive electrode for an electrosurgical handpiece according to an embodiment of the present invention can reduce the degree of fatigue of the operating surgeon by reducing the doctor's tension since conducting high frequency electric energy only to the first blade 101 in order to perform a precise electrosurgery.

Additionally, the conductive electrode for an electrosurgical handpiece according to an embodiment of the present invention can reduce contamination of the blades and a patient's burn at the time of the precise surgery, and does not generate smog which has a bad influence on the operating surgeon's and the patient's health.

1. A conductive electrode for an electrosurgical handpiece, which is coupled to a handpiece used for electrosurgery, the conductive electrode comprising:
a first blade (101) formed of a conductive material in a plate shape;
a second blade (103) formed of a conductive material in a plate shape; and
a gap (A) formed between the first and second blades (101, 103) so that the first and second blade (101, 103) are spaced apart from each other at a predetermined gap.

2. The conductive electrode according to claim 1, further comprising:
a first plug (102) formed to be extended from the rear end of the first blade (101) and inserted and coupled into a handpiece (30); and
a second plug (104) formed to be extended from the rear end of the second blade (103) and inserted and coupled into a handpiece (30).

3. The conductive electrode according to claim 1, wherein a front end portion of the gap (A) has a split angle (α) which is inclined at a predetermined angle to an axial line of the conductive electrode (100).

4. The conductive electrode according to claim 3, wherein the split angle (α) is formed to be inclined in the direction of the first blade (101).

5. The conductive electrode according to claim 1, wherein the split angle (α) is within a range exceeding 0° but not exceeding 120° in the direction of the first blade (101).

6. The conductive electrode according to claim 1, wherein the first blade (101) and the second blade (103) are coated with an insulating material.
